(19) United States
(12) Patent Application Publication
(10) Pub. No.: US 2025/0127837 A1
(43) Pub. Date: Apr. 24, 2025

(54) FERMENTED TRADITIONAL CHINESE MEDICINE COMPOSITE PREPARATION FOR PREVENTING AND TREATING PIGLET VIRAL DIARRHEA, PREPARATION METHOD AND APPLICATION

(71) Applicant: Henan University of Chinese Medicine, Zhengzhou (CN)

(72) Inventors: Shuying Feng, Luoyang (CN); Aifang Li, Zhengzhou (CN); Qianqian Wang, Zhengzhou (CN); Dandan Guo, Zhengzhou (CN); Shuxuan Li, Zhengzhou (CN); Wei Chen, Zhengzhou (CN); Cunhai Hu, Luoyang (CN)

(21) Appl. No.: 18/776,259

(22) Filed: Jul. 18, 2024

(30) Foreign Application Priority Data
Oct. 20, 2023 (CN) 202311366112.4

(51) Int. Cl. A61K 36/71; A61K 35/00; A61K 35/742; A61K 35/747; A61K 36/05; A61K 36/232; A61K 36/28; A61K 36/284; A61K 36/285; A61K 36/31; A61K 36/484; A61K 36/488; A61K 36/575; A61K 36/65; A61K 36/718; A61K 39/42; A61K 47/42; A61P 31/14

(52) U.S. Cl. CPC A61K 36/71; A61K 35/742; A61K 35/747; A61K 36/05; A61K 36/232; A61K 36/28; A61K 36/284; A61K 36/285; A61K 36/31; A61K 36/484; A61K 36/488; A61K 36/575; A61K 36/65; A61K 36/718; A61K 39/42; A61K 47/42; A61P 31/14; A61K 2035/115; A61K 2236/19

(57) ABSTRACT

A fermented traditional Chinese medicine composite preparation for preventing and treating piglet viral diarrhea is prepared by: fermenting a traditional Chinese medicine composition as a basic raw material with five probiotics followed by mixing with a Dunaliella salina liquid, a PEDV yolk antibody, a whey powder, sodium chloride and glucose. The traditional Chinese medicine composition includes following medicinal materials: 1-30 parts of Pulsatilla chinensis, 1-30 parts of Radix paeoniae alba, 1-10 parts of Coptis chinensis, 1-10 parts of Pueraria lobata, 1-15 parts of Bupleurum chinense, 1-20 parts of fried Atractylodes macrocephala, 1-10 parts of Semen raphani, 1-10 parts of Magnolia officinalis, 1-10 parts of Angelica sinensis, 1-10 parts of Aucklandia lappa, and 1-15 parts of Glycyrrhiza uralensis. The traditional Chinese medicine composition is fermented with the five probiotics based on biological fermentation technologies to achieve traditional Chinese medicine biotransformation through microorganism, alter medicine properties, and achieve enhancing efficacy and detoxification.

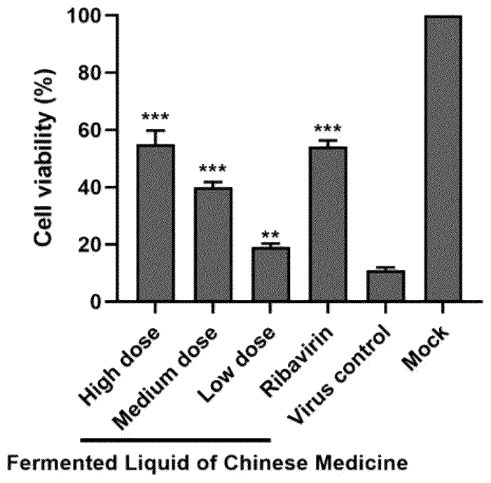

### CROSS-REFERENCE TO RELATED APPLICATION

This application claims priority to Chinese Patent Application No. 202311366112.4, filed on Oct. 20, 2023, which is herein incorporated by reference in its entirety.

### TECHNICAL FIELD

The disclosure relates to the field of probiotic fermentation, and more particularly to a fermented traditional Chinese medicine composite preparation for preventing and treating piglet viral diarrhea, its preparation method and application.

### BACKGROUND

Piglet diarrhea is a common epidemic disease in pig farms, main pathogens causing the viral diarrhea in pigs include porcine epidemic diarrhea virus (PEDV), porcine transmissible gastroenteritis virus, and porcine rotavirus etc., and the piglet diarrhea is often accompanied by co-infection or cross-infection with multiple pathogens. Main clinical symptoms of the piglet diarrhea include vomiting, diarrhea and dehydration, etc. In large-scale breeding farms, the incidence of the piglet diarrhea can be as high as about 57%. Mild diarrhea can lead to indigestion and slow growth in piglets, while severe diarrhea can lead to dehydration and death, with a mortality rate of 70% to 100%. At present, the piglet diarrhea seriously hinders healthy development of the pig breeding industry in China and even the world, causing huge economic losses to the pig breeding industry. However, there are currently no safe and effective specific drugs against the piglet viral diarrhea. Related symptomatic treatment drugs usually have problems such as drug resistance and drug residues, affecting food safety. The trivalent live vaccine targeting the three common virus strains has a disadvantage of being difficult to scale up vaccination and carries a potential risk of causing the virus strains to revert to virulence. Therefore, strengthening feeding management is still a main prevention and control measure, lacking targeted measures with poor effects. There is an urgent need to carry out research on green, environmentally friendly, safe, and effective drugs for preventing and treating the piglet viral diarrhea.

At present, there are multiple research reports on the prevention and treatment of the piglet diarrhea with traditional Chinese medicine. However, raw materials of the traditional Chinese medicine are mostly plants with high fiber content, low bioavailability, and weak pharmacological effects, and have strong woody texture affecting feed palatability, thereby affecting the feed intake of the piglets. The treatment effect needs to be further improved. In order to improve the effectiveness and palatability of traditional Chinese medicine formulations, the development of traditional Chinese medicine microecological preparations has become the main research direction and hot field at present. Probiotics fermented traditional Chinese medicine applies modern fermentation engineering technologies to inoculate beneficial microorganisms into a substrate of the traditional Chinese medicine for growth and reproduction. After fermentation, not only can the palatability be improved, but also the content of effective components and the bioavailability of the traditional Chinese medicine can be increased, enhancing the medicinal effect and reducing toxic side effects. Therefore, the development of fermented traditional Chinese medicine compound feed additives is popular. However, there are currently few patents related to fermented traditional Chinese medicine preparations for preventing and treating the piglet viral diarrhea, indicating a large research and development space.

### SUMMARY

To overcome shortcomings and disadvantages in the related art, a first purpose of the disclosure is to provide a fermented traditional Chinese medicine composite preparation for preventing and treating piglet viral diarrhea.

The fermented traditional Chinese medicine composite preparation uses a traditional Chinese medicine composition as a basic raw material. The traditional Chinese medicine composition includes the following medicinal materials in parts by weight: 1-30 parts of Pulsatilla chinensis, 1-30 parts of Radix paeoniae alba (also referred to as white peony root), 1-10 parts of Coptis chinensis, 1-10 parts of Pueraria lobata, 1-15 parts of Bupleurum chinense, 1-20 parts of fried Atractylodes macrocephala, 1-10 parts of Semen raphanin (also referred to as radish seed), 1-10 parts of Magnolia officinalis, 1-10 parts of Angelica sinensis, 1-10 parts of Aucklandia lappa, and 1-15 parts of Glycyrrhiza uralensis. The traditional Chinese medicine composition is added with water to adjust a water content of the traditional Chinese medicine composition in a range of 30%-50%, and then added with probiotics including: Bacillus subtilis, Bacillus licheniformis, Lactobacillus plantarum, Lactobacillus casei and Clostridium butyricum to obtain a traditional Chinese medicine fermented liquid. When preparing a feed additive, an algal extract with a volume in a range of 1%-10% of a volume of the traditional Chinese medicine fermented liquid, a PEDV yolk antibody with a volume in a range of 1%-10% of the volume of the traditional Chinese medicine fermented liquid, a whey powder with a volume in a range of 1%-10% of the volume of the traditional Chinese medicine fermented liquid and other components (such as a formulating agent and an ingredient etc.) are added to the traditional Chinese medicine fermented liquid to obtain the fermented traditional Chinese medicine composite preparation of the disclosure.

A second purpose of the disclosure is to provide a preparation method of the fermented traditional Chinese medicine composite preparation for preventing and treating piglet viral diarrhea, including following steps:
(1) preparing a traditional Chinese medicine fermentation culture medium: selecting, cleaning and then pulverizing the medicinal materials individually followed by filtering through an 80-mesh sieve to obtain powder of each medicinal material, weighing and adding the powder of each medicinal material in the parts by weight to a mixer followed by mixing for 25-35 minutes (min) to obtain a traditional Chinese medicine composition mixture, adding water with an amount 10-20 times a weight of the traditional Chinese medicine composition mixture to the traditional Chinese medicine composition mixture followed by mixing to obtain a liquid mixture, and sterilizing the liquid mixture at high pressure (0.1 MPa) at a temperature of 121° C. for 30 min followed by cooling to a temperature of 37° C. to obtain the traditional Chinese medicine fermentation culture medium;
(2) activation and scale-up cultivation of fermentation probiotic strains: activating the Bacillus subtilis and the Bacillus licheniformis individually by using Luria-Bertani (LB) liquid culture media and activating the Lactobacillus plantarum, the Lactobacillus casei and the Clostridium butyricum individually by using DeMan, Rogosa and Sharpe (MRS) liquid culture media to obtain a probiotic suspension of each of the Bacillus subtilis, the Bacillus licheniformis, the Lactobacillus plantarum, the Lactobacillus casei and the Clostridium butyricum, measuring a bacterial concentration of each probiotic suspension by a turbidimeter, and adjusting the bacterial concentration of each probiotic suspension to 1×10⁸ colony-forming units per milliliter (CFU/mL) by using the LB liquid culture media and the MRS liquid culture media correspondingly to obtain an adjusted probiotic suspension of each of the Bacillus subtilis, the Bacillus licheniformis, the Lactobacillus plantarum, the Lactobacillus casei and the Clostridium butyricum;
(3) a traditional Chinese medicine fermentation process with the probiotics: adding each adjusted probiotic suspension to the traditional Chinese medicine fermentation culture medium prepared in the step (1) with an addition amount of each adjusted probiotic suspension in a range of 2%-4% of a volume of the traditional Chinese medicine fermentation culture medium to obtain a mixture, sealing the mixture at a temperature of 37° C. for fermentation for 2-7 days to obtain a fermented mixture, stirring the mixture occasionally during the fermentation, and filtering the fermented mixture after the fermentation through an 80-mesh sieve to obtain the traditional Chinese medicine fermented liquid; and
(4) preparation of the fermented traditional Chinese medicine composite preparation: adding the algal extract with a volume in a range of 1%-10% of a volume of the traditional Chinese medicine fermented liquid, the PEDV yolk antibody with a volume in a range of 1%-10% of the volume of the traditional Chinese medicine fermented liquid, the whey powder with a volume in a range of 1%-10% of the volume of the traditional Chinese medicine fermented liquid to the traditional Chinese medicine fermented liquid prepared in the step (3), and then adding sodium chloride and glucose with an appropriate amount to the traditional Chinese medicine fermented liquid to regulate body fluid balance and supply carbon source, followed by mixing to obtain the fermented traditional Chinese medicine composite preparation for preventing and treating the piglet viral diarrhea.

In an embodiment, in the step (1), the pulverizing the medicinal materials includes: pulverizing the medicinal materials by ultrafine pulverization.

In an embodiment, in the step (1), the sterilizing the liquid mixture includes: soaking the liquid mixture for 4-6 hours followed by sterilizing.

In an embodiment, a preparation method of the LB liquid culture media in the step (2) includes: 0.3 grams (g) of beef extract, 1.0 g of peptone, 0.5 g of the sodium chloride, 1.5 g of agar are added with distilled water to 1000 milliliter (mL), and then adjusted to have a potential of hydrogen (pH) in a range of 7.2-7.6, followed by sterilizing at high pressure at a temperature of 121° C. for 20 min.

In an embodiment, a preparation method of the MRS liquid culture media in the step (2) includes: 10.0 g of the beef extract, 5.0 g of yeast extract powder, 20.0 g of the glucose, 10.0 g of the peptone, 2.0 g of dipotassium hydrogen phosphate (K₂HPO₄), 0.5 g of cysteine, 5.0 g of sodium acetate (CH₃COONa), 2.0 g of ammonium citrate dibasic (C₆H₁₄N₂O₇), 0.58 g of magnesium sulfate (MgSO₄), 0.28 g of manganese sulfate (MnSO₄)m and 1.0 g of tween-80 are added with distilled water to 1000 mL, and then adjusted to have a pH in a range of 7.2-7.6 with sodium hydroxide (NaOH) followed by sterilizing at high pressure at a temperature of 121° C. for 20 min.

In an embodiment, in the step (3), time of the fermentation is 4 days.

In an embodiment, in the step (4), an alga for preparing the algal extract includes Dunaliella salina cultured to a logarithmic growth phase, and the algal extract is prepared by: mixing the alga with water to obtain an algal suspension, adjusting a cell wet weight in the algal suspension to a range of 80-200 grams per liter (g/L), and then disrupting alga cells in the algal suspension at high pressure in a range of 80-120 mega Pascals (MPa) followed by centrifuging the algal suspension at a low speed to collect a supernatant to obtain the algal extract.

In an embodiment, a dosage form of the fermented traditional Chinese medicine composite preparation in the step (4) is a formulation for gastrointestinal administration, in a form of liquid, powder, or paste.

A third purpose of the disclosure is to provide an application method of the fermented traditional Chinese medicine composite preparation, including: applying the fermented traditional Chinese medicine composite preparation in preparing drugs for preventing and treating the piglet viral diarrhea.
(1) Preventive method: During an epidemic period of PEDV, the traditional Chinese medicine fermentation composite preparation is applied in feed additives, with an addition amount of 1%-5% of a basic daily ration weight, once a day, continuously for 7-10 days.
(2) Treatment method: when piglets start being infected with the PEDV or when piglets have symptoms of vomiting and diarrhea, the traditional Chinese medicine fermentation composite preparation is applied in feed additives, with an addition amount of 1%-5% of the basic daily ration weight, once a day, continuously for 7 to 10 days, and then continuously for 5 days after the symptom are controlled.

In the disclosure, each component of the traditional Chinese medicine composition can be purchased from commercially available products. Principles of the components in the traditional Chinese medicine composition are as follows. The Pulsatilla chinensis has effects such as clearing heat and detoxifying, as well as cooling blood and stopping dysentery etc. The Radix paeoniae alba has effects such as warming yang and dispelling dampness, tonifying deficiency of the body, strengthening spleen and stomach, astringing yin to stop sweating, and stabilizing liver yang (referring chiefly to the warming, upbearing and coursing-freeing function of the liver) etc. The Coptis chinensis can clear heat, dry dampness, purge fire (a more intense form of heat and often related to acute conditions, irritability, and intense inflammation, purge fire means eliminate these extreme heat conditions) and detoxify, mainly used for symptoms such as damp-heat diarrhea and dysentery, damp-heat of liver and gallbladder and bleeding, etc., and has effects such as antipyretic, detoxifying, enhancing immunity, antibacterial, antiviral, and anti-endotoxin etc. The Pueraria lobata has effects such as relieving fever and muscle pain, promoting fluid production to quench thirst, raising yang to stop diarrhea, dredging meridians and activating collaterals, and detoxifying from alcohol, commonly used for symptoms such as headache and fever caused by external pathogenic invasion, thirst, diabetes, dysentery, diarrhea, dizziness and headache, etc. The Bupleurum chinense has effects of harmonizing the interior and exterior, soothing the liver and raising the yang, and is used for treating conditions such as colds with fever, alternating chills and fever, malaria, liver depression and qi stagnation, and chest and rib distending pain, etc. The fried Atractylodes macrocephala has effects such as strengthening the spleen and benefiting qi (qi refers to a vital energy within the human body, which runs through various parts of the body and supports life activities), and drying dampness and inducing diuresis etc., and is used for symptoms such as spleen deficiency with poor appetite, and abdominal distension and diarrhea, etc. The Semen raphani has efficacy of benefiting qi. When used raw, the Semen raphani can ascend qi to expel wind-phlegm, disperse wind-cold, and promote the eruption of rashes. When cooked, the Semen raphani can descend qi to alleviate cough and asthma caused by phlegm, regulate and tenesmus after dysentery, and relieve internal pain, all of which are manifestations of qi-advancing properties. The Magnolia officinalis has effects such as soothing middle-jiao (upper abdomen including stomach and spleen etc.) and regulating qi, resolving dampness and relieving depression, and eliminating stagnation and relieving asthma etc. The Angelica sinensis is used for symptoms such as deficiency-cold abdominal pain, constipation due to intestinal dryness, tenesmus after bloody dysentery etc. The Aucklandia lappa has effects of moving qi to relieve pain, strengthening spleen and promoting digestion, and is used for symptoms such as tenesmus after diarrhea and dysentery, indigestion with food stagnation and lack of appetite etc. The Glycyrrhiza uralensis has effects such as tonifying spleen to boost qi, reducing toxicity and detoxifying, soothing the middle-jiao, alleviating acute symptoms, and harmonizing the actions of all medicinals in a formula, and is used for symptoms such as spleen-stomach weakness, fatigue, lack of energy, and poisoning etc.

In traditional Chinese veterinary medicine, it is believed that piglets have immature qi and blood, and their bodies are in a state of immature yang. If there is an imbalance in cold and heat, or improper feeding management, it is prone to a dysfunction of the spleen and stomach, thereby causing diarrhea. Generally, main treatment strategies are to remove dampness and stop diarrhea, as well as clear heat and detoxify. A classical prescription of Baitouweng Tang (a decoction with the Pulsatilla chinensis as a main ingredient) for treating the piglet diarrhea is formulated based on the cause and symptoms of the disease. According to the treatment principles, the following herbs are selected: the Pulsatilla chinensis, Phellodendron chinense, the Coptis chinensis, and Fraxinus rhynchophylla to treat conditions caused by heat-toxicity deeply involved the blood aspect and downward pressure on the large intestine, and the main treatment is clearing heat, detoxifying, cooling blood, and stopping dysentery. In the classic prescription, the Pulsatilla chinensis serves as a monarch drug (also referred to as a principal drug or a sovereign drug, an ingredient that provides a principal curative action on a main pattern/syndrome or primary symptom) to clear heat, detoxify, cool blood, and stop dysentery, the Phellodendron chinense serves as a minister drug (an ingredient that helps strengthen the principal curative action) to purge damp-heat from the lower-jiao (refers to the lower part of the body's three energetic compartments, which includes the lower abdomen from the stomach outlet down to the genital and urinary areas), together achieving the effect of drying dampness and stopping dysentery. The Coptis chinensis is bitter and cold, with effects of clearing heat, drying dampness, purging fire and detoxifying. The Fraxinus rhynchophylla is bitter, cold and with astringent properties, having a strong astringent effect. The combination of these four herbs is an excellent prescription for heat toxin and bloody dysentery. The Atractylodes macrocephala can benefit qi and strengthen the spleen, a healthy spleen is fundamental for removing dampness, mainly enhancing disease resistance and immunity of the piglets, achieving the purpose of preventing and treating diseases. The Pulsatilla chinensis serves as the assistant and courier for anti-bacteria and anti-dysentery, with astringent properties to control the intestines and stop diarrhea. The Pulsatilla chinensis can not only control the primary disease but also prevent secondary infections, achieving the combination of prevention and treatment. The composition of the preparation is mutually supportive in pharmacology, and the combination of prescriptions conforms to the principles of compatibility in the traditional Chinese veterinary medicine, having an excellent effect on preventing and treating the piglet diarrhea.

Compared to the related art, the disclosure has following beneficial effects.
(1) The fermented traditional Chinese medicine composite preparation prepared by the disclosure is based on the classical prescription of Baitouweng Tang with modifications made to serve as a fundamental traditional Chinese medicine raw material. The components cooperate with each other to achieve a synergistic effect, enhancing the therapeutic efficacy. Meanwhile, the traditional Chinese medicine composition is fermented with quintuple probiotics to further improve effectiveness of the fermented traditional Chinese medicine composite preparation. Traditional Chinese medicine fermentation, as a processing method and technique in the traditional Chinese medicine, can significantly alter medicine properties, improve the bioavailability, and achieve effects of enhancing efficacy and detoxification.
(2) The fermented traditional Chinese medicine composite preparation prepared by the disclosure utilizes five probiotics including the Bacillus subtilis, the Bacillus licheniformis, the Lactobacillus plantarum, the Lactobacillus casei, and the Clostridium butyricum as fermentation strains, achieving the biotransformation of the traditional Chinese medicine by microorganisms, thereby realizing a synergistic detoxification effect on the components of the traditional Chinese medicine. Moreover, the combined fermentation of multiple probiotics can produce a variety of substances such as probiotic enzymes, amino acids and trace elements etc., regulate the intestinal microecological balance and inhibit the growth and reproduction of pathogenic microorganisms, which are particularly beneficial for preventing and treating the piglet diarrhea.
(3) The disclosure uses the PEDV yolk antibody, significantly improving an antiviral effect, with superior performance than a traditional Chinese medicine preparation with fermentation only.
(4) In the fermented traditional Chinese medicine composite preparation prepared by the disclosure, substances such as Dunaliella salina liquid (i.e., the algal extract), the whey powder, the sodium chloride, and the glucose are added. The Dunaliella salina liquid is rich in vitamins and trace elements, and has a function of protecting the gastrointestinal mucosa and regulating the electrolyte balance. The whey powder can provide a large amount of lactose, which can produce lactic acid when fermented in the piglet digestive tract, helping digestion of the piglets and providing high-quality whey protein nutrients. The sodium chloride and the glucose mainly provide energy, regulate fluid balance and improve taste. The addition of the substances synergistically improves the therapeutic effect of the fermented traditional Chinese medicine composite preparation, accelerates the disease recovery process, and shortens the medication time.

### BRIEF DESCRIPTION OF DRAWING

FIGURE illustrates an in vitro anti-PEDV activity analysis result diagram of a fermented traditional Chinese medicine composite preparation.

### DETAILED DESCRIPTION OF EMBODIMENTS

A further detailed description of the disclosure is provided below in conjunction with embodiments, but the embodiments of the disclosure are not limited to these. In the following embodiments, unless otherwise specified, methods in the embodiments are all conventional methods, and materials and reagents etc., used are obtained from commercial sources or prepared by using conventional methods.

### Embodiment 1: Preparation of a Fermented Traditional Chinese Medicine Composite Preparation for Preventing and Treating Piglet Viral Diarrhea in a Liquid Form

(1) Preparation of a traditional Chinese medicine fermentation culture medium: the following medicinal materials are weighed in parts by weight: 1-30 parts of Pulsatilla chinensis, 1-30 parts of Radix paeoniae alba, 1-10 parts of Coptis chinensis, 1-10 parts of Pueraria lobata, 1-15 parts of Bupleurum chinense, 1-20 parts of fried Atractylodes macrocephala, 1-10 parts of Semen raphani, 1-10 parts of Magnolia officinalis, 1-10 parts of Angelica sinensis, 1-10 parts of Aucklandia lappa and 1-15 parts of Glycyrrhiza uralensis. The medicinal materials are then selected, cleaned and pulverized individually followed by filtering through an 80-mesh sieve to obtain powder of each medicinal material, the powder of each medicinal material are added to a mixer followed by mixing for 25-35 min to obtain a traditional Chinese medicine composition mixture, water with an amount 15 times a weight of the traditional Chinese medicine composition mixture (volume to mass abbreviated as v/m) is added to the traditional Chinese medicine composition mixture followed by mixing to obtain a liquid mixture, and the liquid mixture is sterilized at high pressure at a temperature of 121° C. for 30 min followed by cooling to a temperature of 37° C. to obtain the traditional Chinese medicine fermentation culture medium.
(2) Activation and scale-up cultivation of fermentation probiotic strains: five probiotics including Bacillus subtilis, Bacillus licheniformis, Lactobacillus plantarum, Lactobacillus casei and Clostridium butyricum are activated individually by using corresponding liquid culture media to obtain a probiotic suspension of each of the Bacillus subtilis, the Bacillus licheniformis, the Lactobacillus plantarum, the Lactobacillus casei and the Clostridium butyricum. A bacterial concentration of each probiotic suspension is measured by a turbidimeter, and the bacterial concentration of each probiotic suspension is adjusted to 1×10⁸ CFU/mL by using the corresponding culture media to obtain an adjusted probiotic suspension of each of the Bacillus subtilis, the Bacillus licheniformis, the Lactobacillus plantarum, the Lactobacillus casei and the Clostridium butyricum.
(3) A traditional Chinese medicine fermentation process with the probiotics: each adjusted probiotic suspension is added to the traditional Chinese medicine fermentation culture medium prepared in the step (1) with an addition amount of each adjusted probiotic suspension of 2% (v/m) of a volume of the traditional Chinese medicine fermentation culture medium to obtain a mixture, the mixture is sealed at a temperature of 37° C. for fermentation for 2 days to obtain a fermented mixture, the mixture is stirred occasionally during the fermentation, and the fermented mixture is filtered after the fermentation through an 80-mesh sieve to obtain a traditional Chinese medicine fermented liquid.
(4) Preparation of the fermented traditional Chinese medicine composite preparation: an algal extract liquid with a volume of 1% of a volume of the traditional Chinese medicine fermented liquid, a porcine yolk antibody liquid with a volume of 5% of the volume of the traditional Chinese medicine fermented liquid, a whey powder with a volume of 1% of the volume of the traditional Chinese medicine fermented liquid are added to the traditional Chinese medicine fermented liquid prepared in the step (3), and then 0.1% of sodium chloride and 0.1% of glucose are added to the traditional Chinese medicine fermented liquid followed by mixing thoroughly to obtain the fermented traditional Chinese medicine composite preparation for preventing and treating the piglet viral diarrhea.
(5) The fermented traditional Chinese medicine composite preparation is in an oral liquid form, which is suitable for direct drinking or gavage administration for animals.

### Embodiment 2: Preparation of a Fermented Traditional Chinese Medicine Composite Preparation for Preventing and Treating Piglet Viral Diarrhea in a Powder Form

(1) Preparation of a traditional Chinese medicine fermentation culture medium: the following medicinal materials are weighed in parts: 1-30 parts of Pulsatilla chinensis, 1-30 parts of Radix paeoniae alba, 1-10 parts of Coptis chinensis, 1-10 parts of Pueraria lobata, 1-15 parts of Bupleurum chinense, 1-20 parts of fried Atractylodes macrocephala, 1-10 parts of Semen raphani, 1-10 parts of Magnolia officinalis, 1-10 parts of Angelica sinensis, 1-10 parts of Aucklandia lappa, and 1-15 parts of Glycyrrhiza uralensis, the medicinal materials are then selected, cleaned and pulverized individually followed by filtering through an 80-mesh sieve to obtain powder of each medicinal material, the powder of each medicinal material are added to a mixer followed by mixing for 25-35 min to obtain a traditional Chinese medicine composition mixture, water with an amount 15 times a weight of the traditional Chinese medicine composition mixture (v/m) is added to the traditional Chinese medicine composition mixture followed by mixing thoroughly to obtain a liquid mixture, and the liquid mixture is sterilized at high pressure at a temperature of 121° C. for 30 min followed by cooling to a temperature of 37° C. to obtain the traditional Chinese medicine fermentation culture medium.
(2) Activation and scale-up cultivation of fermentation probiotic strains: five probiotics including Bacillus subtilis, Bacillus licheniformis, Lactobacillus plantarum, Lactobacillus casei and Clostridium butyricum are activated individually by using corresponding liquid culture media to obtain a probiotic suspension of each of the Bacillus subtilis, the Bacillus licheniformis, the Lactobacillus plantarum, the Lactobacillus casei and the Clostridium butyricum, a bacterial concentration of each probiotic suspension are measured by a turbidimeter, and the bacterial concentration of each probiotic suspension is adjusted to 1×10⁸ CFU/mL by using the corresponding culture media to obtain an adjusted probiotic suspension of each of the Bacillus subtilis, the Bacillus licheniformis, the Lactobacillus plantarum, the Lactobacillus casei and the Clostridium butyricum.
(3) A traditional Chinese medicine fermentation process with the probiotics: each adjusted probiotic suspension is added to the traditional Chinese medicine fermentation culture medium prepared in the step (1) to obtain a mixture, an addition amount of each adjusted probiotic suspension is correspondingly 3%, 2%, 2%, 2% and 1% (v/m) of a volume of the traditional Chinese medicine fermentation culture medium, the mixture is sealed at a temperature of 37° C. for fermentation for 4 days to obtain a fermented mixture, the mixture is stirred occasionally during the fermentation for substance dissolution, and the fermented mixture is filtered after the fermentation through an 80-mesh sieve to obtain a traditional Chinese medicine fermented liquid. The traditional Chinese medicine fermented liquid is added with 10% of hydroxypropyl-β-cyclodextrin and then freeze-dried at a low-temperature to obtain freeze-dried traditional Chinese medicine fermented powder.
(4) Preparation of the fermented traditional Chinese medicine composite preparation: 5% of a whey powder, 1% of a porcine yolk antibody powder, 2% of a Dunaliella salina powder, 0.5% of sodium chloride and 0.5% of glucose are added to the freeze-dried traditional Chinese medicine fermented powder to obtain the fermented traditional Chinese medicine composite preparation for preventing and treating the piglet viral diarrhea.
(5) The fermented traditional Chinese medicine composite preparation for preventing and treating the piglet viral diarrhea is in a powder form, which can be added to drinking water or liquid milk powder for use, and should be consumed in one sitting.

### Embodiment 3: Preparation of a Fermented Traditional Chinese Medicine Composite Preparation for Preventing and Treating Piglet Viral Diarrhea in a Paste Form

(1) Preparation of a traditional Chinese medicine fermentation culture medium: the following medicinal materials are weighed in parts: 1-30 parts of Pulsatilla chinensis, 1-30 parts of Radix paeoniae alba, 1-10 parts of Coptis chinensis, 1-10 parts of Pueraria lobata, 1-15 parts of Bupleurum chinense, 1-20 parts of fried Atractylodes macrocephala, 1-10 parts of Semen raphani, 1-10 parts of Magnolia officinalis, 1-10 parts of Angelica sinensis, 1-10 parts of Aucklandia lappa and 1-15 parts of Glycyrrhiza uralensis, the medicinal materials are then selected, cleaned and pulverized individually followed by filtering through an 80-mesh sieve to obtain a powder of each medicinal material, the powder of each medicinal material are added to a mixer followed by mixing for 25-35 min to obtain a traditional Chinese medicine composition mixture, water with an amount 15 times a weight of the traditional Chinese medicine composition mixture (v/m) is added to the traditional Chinese medicine composition mixture followed by mixing thoroughly to obtain a liquid mixture, and the liquid mixture is sterilized at high pressure at a temperature of 121° C. for 30 min followed by cooling to a temperature of 37° C. to obtain the traditional Chinese medicine fermentation culture medium.
(2) Activation and scale-up cultivation of fermentation probiotic strains: five probiotics including Bacillus subtilis, Bacillus licheniformis, Lactobacillus plantarum, Lactobacillus casei and Clostridium butyricum are activated individually by using corresponding liquid culture media to obtain a probiotic suspension of each of the Bacillus subtilis, the Bacillus licheniformis, the Lactobacillus plantarum, the Lactobacillus casei and the Clostridium butyricum, a bacterial concentration of each probiotic suspension are measured by a turbidimeter, and the bacterial concentration of each probiotic suspension is adjusted to 1×10⁸ CFU/mL by using the corresponding culture media to obtain an adjusted probiotic suspension of each of the Bacillus subtilis, the Bacillus licheniformis, the Lactobacillus plantarum, the Lactobacillus casei and the Clostridium butyricum.
(3) A traditional Chinese medicine fermentation process with the probiotics: each adjusted probiotic suspension is added to the traditional Chinese medicine fermentation culture medium prepared in the step (1) to obtain a mixture, an addition amount of each adjusted probiotic suspension is correspondingly 2.5%, 2.5%, 2.5%, 2.5% and 1% (v/m) of a volume of the traditional Chinese medicine fermentation culture medium, the mixture is sealed at a temperature of 37° C. for fermentation for 7 days to obtain a fermented mixture, the mixture is stirred occasionally during the fermentation for substance dissolution, and the fermented mixture is filtered after the fermentation through an 80-mesh sieve to obtain a traditional Chinese medicine fermented liquid.
(4) The traditional Chinese medicine fermented liquid is concentrated into a semi-fluid paste by a low-temperature freeze-drying process, with water content controlled in a range of 15%-20%.
(5) Preparation of the fermented traditional Chinese medicine composite preparation in the paste form: 10% of a whey powder, 5% of a porcine yolk antibody powder, 5% of a Dunaliella salina powder, 1% of sodium chloride and 1% of glucose are added to the semi-fluid paste and then mixed thoroughly to obtain the fermented traditional Chinese medicine composite preparation in the paste form for preventing and treating the piglet viral diarrhea.
(6) The fermented traditional Chinese medicine composite preparation is in the paste form or in a semi-fluid form, which can be directly fed or mixed with drinking water or liquid milk powder when used.

### Effect Embodiment 1: An Analysis of In Vitro Anti-PEDV Activity of the Fermented Traditional Chinese Medicine Composite Preparation for Preventing and Treating the Piglet Viral Diarrhea in the Liquid Form

The in vitro anti-PEDV activity of the fermented traditional Chinese medicine composite preparation described in the disclosure is detected by using a cell counting kit-8 (CCK-8) method. The CCK-8 method is used to detect an optical density value at 450 nanometers (OD_{450 nm} value) of each well according to an instruction of a reagent kit to calculate a cell survival rate. The experiment sets 6 groups including an embodiment 1 group with a high drug concentration (1600 micrograms per milliliter, abbreviated as μg/mL), an embodiment 2 group with a medium drug concentration (100 μg/mL), an embodiment 3 group with a low drug concentration (3.125 μg/mL), a ribavirin drug group (25 μg/mL), a PEDV control group and a normal cell control group (Mock group). Each group has 3 replicate wells.

A specific operation method is as follows: Vero cells with good growth status are taken and spread onto a 96-well plate (with a cell count of 1×10⁴ per well). After the Vero cells adhere to well walls, the wells in the groups are added with corresponding drugs according to the above concentration gradient. After 2 hours, a PEDV culture medium (multiplicity of infection is 0.1, i.e., MOI=0.1) is added to the Vero cells, and no virus is added in the Mock group. Then, the 96-well plate is placed in an incubator at 37° C. for culture for 48 hours. A cell pathology situation is observed under a microscope, and the cell survival rate is detected by using the CCK-8 method.

The experimental results are shown in FIGURE. Compared with the PEDV control group, the fermented traditional Chinese medicine composite preparations prepared in the embodiment 1 group, the embodiment 2 group and the embodiment 3 group, as well as the ribavirin control group, can effectively inhibit virus replication. Moreover, the cell survival rate in a high-dose group is significantly higher than that in a low-dose group (i.e., the embodiment 1 group has the best antiviral activity). It can be seen that the fermented traditional Chinese medicine composite preparation of the disclosure has a significant inhibitory effect on PEDV virus infection and a dose-dependent property.

### Effect Embodiment 2: The Effect of the Fermented Traditional Chinese Medicine Composite Preparation on Growth Performance and Prevention of Diarrhea Rate of Healthy Piglets

This experiment is conducted in a medium-sized pig farm in northern Henan, China. A total of 120 piglets which are healthy and unweaned are selected, and each piglet is aged 15±1 days with a body weight of 4.0±0.5 kilograms (kg). The piglets are randomly divided into four groups: a healthy control group, a traditional Chinese medicine composition only group, a quintuple probiotic only group, and a composite preparation group, with three replicates per group and ten piglets per replicate. The healthy control group is fed with milk powder, while the quintuple probiotic only group and the composite preparation group (the fermented traditional Chinese medicine composite preparation in the embodiment 2) are given 9-15 ml of a corresponding preparation per piglet per day, followed by supplementary feeding with an appropriate amount of the milk powder. The traditional Chinese medicine composition only group is supplemented with 9-15 ml of unfermented extract of the traditional Chinese medicine composition per piglet per day in the milk powder. The quintuple probiotic group has a probiotic mixture collected after the fermentation added to the milk powder. The composite preparation group has the fermented traditional Chinese medicine composite preparation added to the milk powder. The experiment is conducted in a same pig house, and all participating piglets are tagged with ear tags. The mental state of each piglet is observed and the growth performance and diarrhea-related indicators are monitored for 28 consecutive days. During this period, the piglets have free access to water and food. At 9:00 and 15:00 every day, a diarrhea condition of the piglets is observed, and the number of piglets with diarrhea is recorded. A diarrhea instance is counted per pig per day, and the diarrhea rate is calculated at the end of the experiment. At the same time, a diarrhea index is scored according to Table 1. An average diarrhea index=sum of diarrhea scores/(the piglet number per group×a total number of experimental days). The diarrhea rate (%)=a total number of diarrhea occurrences/(the piglet number per group×the total number of experimental days)×100%. Meanwhile, feed intake is recorded by pen throughout the experiment, and each piglet is weighed on an empty stomach at 7:00 on the 1^{st} day and the 28^{th} days to calculate the average daily gain, average daily feed intake, and feed-to-gain ratio from the 1st day to the 28^{th} days. The average daily gain=a total weight gain per pen/(the piglet number per pen×the total number of experimental days), the average daily feed intake=a total feed intake per pen/(the piglet number per pen×the total number of experimental days), the feed-to-gain ratio=the average daily feed intake/the average daily gain.

TABLE 1
piglet diarrhea index scoring criteria.

| Diarrhea severity | Fecal shape | Score |
|---|---|---|
| Normal | Strip or granular | 0 |
| Mild | Soft feces, able to form | 1 |
| Moderate | Paste-like, unformed | 2 |
| Sever | Liquid, unformed, feces separated from water | 3 |

The growth performance of each group of the piglets is shown in Table 2. The fermented traditional Chinese medicine composite preparation significantly increases the daily weight gain (P<0.05) and daily feed intake (P<0.05) of the unweaned piglets, reduces the feed-to-gain ratio (P<0.05), promotes growth of the piglets, and helps to improve production performance of the piglets. The fermented traditional Chinese medicine composite preparation has a good trend in improving the growth rate of the unweaned piglets and a good advantage in weight gain during the stage, with a significant preventive and therapeutic effect in preventing digestive tract diseases in the piglets.

TABLE 2
the growth performance of each group of the unweaned piglets.

| | | Traditional | | |
|---|---|---|---|---|
| | | Chinese | | |
| | Healthy | medicine | Quintuple | Composite |
| | control | composition | probiotic | preparation |
| Group | group | only group | only group | group |
| Average daily | 265 ± 0.53 | 274 ± 0.62 | 268 ± 0.37 | 295 ± 0.84 |
| feed intake (g) | | | | |
| Average daily | 212 ± 0.02 | 229 ± 0.15 | 218 ± 1.36 | 254 ± 1.50 |
| gain (g) | | | | |
| Feed-to-gain | 1.25 ± 0.03 | 1.20 ± 0.22 | 1.23 ± 0.06 | 1.16 ± 0.98 |
| ratio | | | | |

The diarrhea situation of the piglets in each group is shown in Table 3. Compared with the healthy control group, the composite preparation group (the fermented traditional Chinese medicine composite preparation) can significantly reduce the diarrhea rate and severe diarrhea rate of the unweaned piglets, with the diarrhea rate dropping by nearly 7%. Moreover, there are no severe watery diarrhea and diseased piglet deaths in all the three replicates of the composite preparation group. Therefore, the fermented traditional Chinese medicine composite preparation of the disclosure can improve the survival rate of the unweaned piglets and has a significant clinical effect on the prevention and alleviation of diarrhea in the unweaned piglets.

TABLE 3
the diarrhea rate (%) at two stages of the piglets in each group

| | | Healthy | Traditional Chinese | Quintuple | Composite |
|---|---|---|---|---|---|
| | Observation | control | medicine composition | probiotic | preparation |
| Stage | indicators | group | only group | only group | group |
| Day 1- | Diarrhea rate | 13.3% ± 0.06 | 9.79% ± 0.27 | 9.94% ± 0.63 | 6.52% ± 0.26 |
| day 14 | Severe diarrhea rate | 6.47% ± 0.79 | 5.22 ± 0.28 | 6.13 ± 0.57 | 0 |
| Day 14- | Diarrhea rate | 9.20% ± 0.32 | 6.53% ± 0.41 | 6.68 ± 0.32 | 1.35% ± 0.27 |
| day 28 | Severe diarrhea rate | 3.31% ± 0.77 | 0 | 0 | 0 |

### Effect Embodiment 3: The Effect of the Fermented Traditional Chinese Medicine Composite Preparation on the Diarrhea Cure Rate in Clinical Diarrhea Piglets

This experiment is conducted in a medium-sized pig farm in northern Henan, China. A total of 120 unweaned diarrhea piglets are selected and each unweaned diarrhea piglet is aged 15±1 days with a body weight of 4.0±0.5 kg. The unweaned diarrhea piglets are randomly divided into four groups: an untreated control group, a traditional Chinese medicine composition only group, a quintuple probiotic only group, and a composite preparation group, with three replicates per group and ten piglets per replicate. Additionally, 30 healthy unweaned piglets with the same age are selected as a healthy control group and a body weight of each healthy unweaned piglet is 4.0±0.5 kg, with three replicates and ten piglets per replicate. The untreated control group and the healthy control group are fed with milk powder, the traditional Chinese medicine composition only group is supplemented with unfermented extract of the traditional Chinese medicine composition in milk powder, at a dosage of 9-15 mL per piglet per day, the quintuple probiotic only group is supplemented with a probiotic combination in milk powder, the composite preparation group is supplemented with the fermented traditional Chinese medicine composite preparation in the embodiment 2 in milk powder. The experiment is conducted in a same pig house and monitored continuously for 28 days, during which the piglets can drink and cat freely.

The diarrhea situation of the piglets is observed every day, and a number of diarrhea piglets is recorded. A diarrhea instance is counted per pig per day, and after the experiment, the diarrhea rate is calculated, and the diarrhea index is scored according to the Table 1. The diarrhea rate=a total number of diarrhea instances/(a number of the piglets in the experiment×30 days)×100%.

A total effective rate includes three aspects: cure, effectiveness, and ineffectiveness.

Criteria for the cure: piglets stop symptoms of vomiting and diarrhea, subsequent feces are normal, appetite of the piglets recovers, and the mental state and activity of the piglets return to normal.

Criteria for the effectiveness: in addition to complete cure, the degree of vomiting and diarrhea of piglets is reduced and piglet appetite increases after treatment, but the piglets do not return to the food intake level of the healthy control group.

Criteria for the ineffectiveness: during the experiment, piglets show no improvement or show death after treatment, and autopsy reveals apparent lesions.

The effective rate=a number of effectively treated piglets/a total number of piglets×100%, and the cure rate=a number of cured piglets/the total number of piglets×100%.

Upon observation, the results are as shown in Table 4. The piglets of the healthy control group do not show symptoms of vomiting and diarrhea, while piglets of the untreated control group with diarrhea symptoms without treatment have a mortality rate about 33%, and the health condition of the remaining piglets does not improve. However, the traditional Chinese medicine composition only group, the quintuple probiotic only group and the composite preparation group show cures. The traditional Chinese medicine composition only group has a higher cure rate and effectiveness rate for the diarrhea piglets than the quintuple probiotic only group, while the composite preparation group has the best treatment effect among the three groups. The symptoms of vomiting and diarrhea in the piglets of the composite preparation group are significantly relieved, and piglet appetite significantly increases, with an effectiveness rate of 96%, indicating that the composite preparation group has the best effect in treating the piglet diarrhea.

TABLE 4
therapeutic effect evaluation of the fermented traditional Chinese
medicine composite preparation on the piglet diarrhea.

| | Cure | Effective | Ineffective | Death |
|---|---|---|---|---|
| Group | rate (%) | rate (%) | rate (%) | rate (%) |
| Healthy control group | 0 | 0 | 0 | 0 |
| Untreated control group | 0 | 0 | 67 ± 2.50 | 33 ± 3.00 |
| Traditional Chinese | 38 ± 2.60 | 67 ± 2.35 | 17 ± 1.90 | 16 ± 1.60 |
| medicine composition | | | | |
| only group | | | | |
| Quintuple probiotic only | 8 ± 1.50 | 42 ± 3.00 | 33 ± 2.81 | 25 ± 2.39 |
| group | | | | |
| Composite preparation | 60 ± 2.31 | 96 ± 1.24 | 0 | 4 ± 0.50 |
| group | | | | |

Above results indicate that the fermented traditional Chinese medicine composite preparation prepared by the disclosure can significantly improve the diarrhea situation of the piglets and increase the feed-to-gain ratio of piglets.

The above is only specific embodiments of the disclosure, but the scope of protection of the disclosure is not limited to this. Although the disclosure is described in detail with reference to the embodiments, it is still possible for those skilled in the art to modify or equivalently replace the technical solutions recorded in the embodiments. Any modifications, equivalent substitutions and improvements etc., made within the spirit and principles of the disclosure shall be covered within the scope of protection of the disclosure. The scope of protection of the disclosure shall be based on the scope of protection of the claims.

What is claimed is:

1. A fermented traditional Chinese medicine composite preparation for preventing and treating piglet viral diarrhea, prepared by fermenting a traditional Chinese medicine composition as a basic raw material with probiotics,
wherein the traditional Chinese medicine composition comprises the following medicinal materials in parts by weight: 1-30 parts of Pulsatilla chinensis, 1-30 parts of Radix paeoniae alba, 1-10 parts of Coptis chinensis, 1-10 parts of Pueraria lobata, 1-15 parts of Bupleurum chinense, 1-20 parts of fried Atractylodes macrocephala, 1-10 parts of Semen raphani, 1-10 parts of Magnolia officinalis, 1-10 parts of Angelica sinensis, 1-10 parts of Aucklandia lappa, and 1-15 parts of Glycyrrhiza uralensis; and
wherein the probiotics comprise: Bacillus subtilis, Bacillus licheniformis, Lactobacillus plantarum, Lactobacillus casei and Clostridium butyricum.

2. The fermented traditional Chinese medicine composite preparation as claimed in claim 1, further comprising: an algal extract, a porcine epidemic diarrhea virus (PEDV) yolk antibody, a whey powder, sodium chloride, and glucose.

3. The fermented traditional Chinese medicine composite preparation as claimed in claim 1, wherein a dosage form of the fermented traditional Chinese medicine composite preparation is a formulation for gastrointestinal administration, in a form of liquid, powder, or paste.

4. A feed additive, comprising the fermented traditional Chinese medicine composite preparation as claimed in claim 1.

5. A preparation method of the fermented traditional Chinese medicine composite preparation as claimed in claim 1, comprising following steps:
(1) preparing a traditional Chinese medicine fermentation culture medium: selecting, cleaning and then pulverizing the medicinal materials individually followed by filtering through an 80-mesh sieve to obtain powder of each of the medicinal materials, weighing and adding the powder of each of the medicinal materials in the parts by weight to a mixer followed by mixing for 25-35 minutes (min) to obtain a traditional Chinese medicine composition mixture, adding water with an amount 10-20 times a weight of the traditional Chinese medicine composition mixture to the traditional Chinese medicine composition mixture followed by mixing to obtain a liquid mixture, and sterilizing the liquid mixture at a pressure at a temperature of 121° C. for 30 min followed by cooling to a temperature of 37° C. to obtain the traditional Chinese medicine fermentation culture medium;
(2) activation and scale-up cultivation of fermentation probiotic strains: activating the Bacillus subtilis and the Bacillus licheniformis individually by using Luria-Bertani (LB) liquid culture media and activating the Lactobacillus plantarum, the Lactobacillus casei and the Clostridium butyricum individually by using DeMan, Rogosa and Sharpe (MRS) liquid culture media to obtain a probiotic suspension of each of the Bacillus subtilis, the Bacillus licheniformis, the Lactobacillus plantarum, the Lactobacillus casei and the Clostridium butyricum, measuring a bacterial concentration of each probiotic suspension by a turbidimeter, and adjusting the bacterial concentration of each probiotic suspension to 1×10⁸ colony-forming units per milliliter (CFU/mL) by using the LB liquid culture media and the MRS liquid culture media correspondingly to obtain an adjusted probiotic suspension of each of the Bacillus subtilis, the Bacillus licheniformis, the Lactobacillus plantarum, the Lactobacillus casei and the Clostridium butyricum;
(3) a traditional Chinese medicine fermentation process with the probiotics: adding each adjusted probiotic suspension to the traditional Chinese medicine fermentation culture medium prepared in the step (1) with an addition amount of each adjusted probiotic suspension in a range of 2%-4% of a volume of the traditional Chinese medicine fermentation culture medium to obtain a mixture, sealing the mixture at a temperature of 37° C. for fermentation for 2-7 days to obtain a fermented mixture, stirring the mixture occasionally during the fermentation, and filtering the fermented mixture after the fermentation through an 80-mesh sieve to obtain a traditional Chinese medicine fermented liquid; and
(4) preparation of the fermented traditional Chinese medicine composite preparation: adding an algal extract with a volume in a range of 1%-10% of a volume of the traditional Chinese medicine fermented liquid, a PEDV yolk antibody with a volume in a range of 1%-10% of the volume of the traditional Chinese medicine fermented liquid, a whey powder with a volume in a range of 1%-10% of the volume of the traditional Chinese medicine fermented liquid, sodium chloride and glucose to the traditional Chinese medicine fermented liquid prepared in the step (3) followed by mixing to obtain the fermented traditional Chinese medicine composite preparation for preventing and treating the piglet viral diarrhea.

6. The preparation method as claimed in claim 5, wherein in the step (1), the pulverizing the medicinal materials comprises: pulverizing the medicinal materials by ultrafine pulverization, and the sterilizing the liquid mixture comprises: soaking the liquid mixture for 4-6 hours followed by sterilizing.

7. The preparation method as claimed in claim 5, wherein in the step (3), time of the fermentation is 4 days.

8. The preparation method as claimed in claim 5, wherein in the step (4), an alga for preparing the algal extract comprises Dunaliella salina cultured to a logarithmic growth phase, and the algal extract is prepared by:
mixing the alga with water to obtain an algal suspension, adjusting a cell wet weight in the algal suspension to a range of 80-200 grams per liter (g/L), and then disrupting alga cells in the algal suspension at high pressure in a range of 80-120 mega Pascals (MPa) followed by centrifuging the algal suspension to collect a supernatant to obtain the algal extract.

9. An application method of the fermented traditional Chinese medicine composite preparation as claimed in claim 1, comprising: applying the fermented traditional Chinese medicine composite preparation in preparing drugs for preventing and treating the piglet viral diarrhea.
